(19) United States
(12) Patent Application Publication
(10) Pub. No.: US 2024/0157014 A1
(43) Pub. Date: May 16, 2024

(54) METHOD AND SYSTEM OF CONTROLLING A NON-HOMOGENEOUS COHORT OF OZONE GAS GENERATING DEVICES

(71) Applicant: 13482073 CANADA INC., MARKHAM (CA)

(72) Inventors: PETER BIRCH, MARKHAM (CA); CHING KUO PAI, MARKHAM (CA)

(21) Appl. No.: 18/418,685

(22) Filed: Jan. 22, 2024

Related U.S. Application Data
Application No. 17/737,330 is a continuation-in-part of application No. 17/718,404, filed on Apr. 12, 2022.
Application No. 17/718,404 is a continuation-in-part of application No. 17/699,488, filed on Mar. 21, 2022.
Application No. 17/699,488 is a continuation of application No. 17/737,330, filed on May 5, 2022.

(51) Int. Cl. A61L 9/015; G07C 9/32; G08B 21/18

(52) U.S. Cl. CPC A61L 9/015; G07C 9/32; G08B 21/182; A61L 9/20

(57) ABSTRACT

A method and system of controlling a non-homogeneous cohort of ozone gas generating devices. The method comprises identifying, at a computing device, a plurality of ozone gas generating devices that constitute the non-homogeneous cohort, the plurality including at least a ceiling-mounted, an in-duct mounted and a portable ozone gas generating devices; continuously detecting, via at least one remote ozone gas sensor device located in a spatial area associated with the non-homogeneous cohort, in conjunction with one or more processors of the computing device, a concentration of ozone gas constituent within ozonated air of the spatial area; and instructing, by the one or more processors responsive to continuously detecting the concentration of the ozone gas constituent as being one of above and below a predetermined threshold concentration, at least one ozone gas generating device of the non-homogeneous cohort to increase or decrease rate of ozone gas generation associated therewith.

### RELATED APPLICATIONS

This application is a Continuation-in-Part of, and claims the benefit of priority to, U.S. patent application Ser. No. 17/699,488 filed on Mar. 21, 2022 and U.S. patent application Ser. No. 17/718,404 filed on Apr. 12, 2022. Said U.S. patent application Ser. Nos. 17/699,488 and 17/718,404 are hereby incorporated by reference in their entirety.

### TECHNICAL FIELD

The disclosure herein relates to controlling ozone gas generating devices, including cloud communication network-based control thereof.

### BACKGROUND

Ozone, a trace gas in the earth's atmosphere, is formed by molecules made up of 3 oxygen atoms (O₃) and has the characteristic of being a powerful oxidizing agent proven to be highly effective in killing bacteria, fungi and molds and inactivating viruses. Ozone can be used for the treatment of potentially contaminated surfaces, water, and ambient air thanks to its powerful germicidal effect on a wide spectrum of microorganisms. Ozone created by various kinds of ozone generators can reach every corner of the environment of a single room or a larger space, without leaving any undesired residues. The effectiveness of ozone in treating microorganisms, especially bacteria and viruses is related to various factors, such as ozone concentration, the temperature of the environment, humidity of the environment and exposure time.

### BRIEF DESCRIPTION OF THE DRAWINGS

FIG. 1 illustrates, in an example embodiment, a ceiling-mounted ozone gas generating device.

FIG. 2 illustrates, in an example embodiment, an in-duct mounted ozone gas generating device.

FIG. 3 illustrates an example embodiment system of a non-homogeneous cohort of ozone gas generating devices deployed in a communication network-based ozone generating system.

FIG. 4 illustrates, in an example embodiment, a computing device architecture for controlling a cohort of ozone gas generating devices.

FIG. 5 illustrates, in an example embodiment, a method of operation in cloud-based control of a non-homogeneous cohort of ozone gas generating devices.

### DETAILED DESCRIPTION

Embodiments herein recognize the need for advantageously leveraging the anti-viral and anti-microbial attributes of ozone gas within an at least partially closed environment of living space, while controlling ozone gas concentration within acceptable levels in order to avoid adverse effects on human beings and other living creatures. Embodiments herein also recognize the need for ozone gas generators of varying types, ozone generation capacities and physical form factors to be incorporated into an optimized and efficient control scheme for deployment across a broad range of building infrastructures, including such as a cloud-based Internet of Things (IoT) networked configuration, able to operationally ramp up and swiftly attain desired ozone gas concentration levels in a given living space, without compromising safety of any beings occupying that living space. Accordingly, embodiments herein provide for such a non-homogeneous cohort of ozone gas generating devices capable of operating in rooms, buildings and other enclosed or partially enclosed habitation spaces.

Provided is a method of generating ozone gas. The method comprises identifying, at a computing device, a plurality of ozone gas generating devices that constitute a cohort, the plurality including at least a ceiling-mounted, an in-duct mounted and a portable ozone gas generating devices; continuously detecting, via at least one remote ozone gas sensor device located in a spatial area associated with the cohort, in conjunction with one or more processors of the computing device, a concentration of ozone gas constituent within ozonated air of the spatial area; and instructing, by the one or more processors responsive to continuously detecting the concentration of the ozone gas constituent as being one of above and below a predetermined threshold concentration, at least one ozone gas generating device of the cohort to increase or decrease rate of ozone gas generation associated therewith.

Also provided is an ozone gas generating system comprising a processor and a non-transitory memory including instructions. The instructions when executed by the processor causes the processor to perform operations comprising identifying, at a computing device, a plurality of ozone gas generating devices that constitute the cohort, the plurality including at least a ceiling-mounted, an in-duct mounted and a portable ozone gas generating devices; continuously detecting, via at least one remote ozone gas sensor device located in a spatial area associated with the cohort in conjunction with one or more processors of the computing device, a concentration of ozone gas constituent within ozonated air of the spatial area; and instructing, by the one or more processors responsive to continuously detecting the concentration of the ozone gas constituent as being one of above and below a predetermined threshold concentration, at least one ozone gas generating device of the cohort to increase or decrease rate of ozone gas generation associated therewith. Generating the ozone gas produces a modified air stream constituted of ozone-rich air, also referred to herein as “ozonated air” which has a higher concentration of ozone gas as compared with ambient air. In embodiments, a network of ozone gas generating devices can be subjected to control using an artificial intelligence or machine learning neural network model, once that model is appropriately trained. In some embodiments, the control scheme can be implemented via a cloud-based server computing device, with individual ozone gas generating devices considered as respective Internet of Things (IoT) nodes within a given ad hoc network.

Also provided is a non-transitory computer-readable memory storing instructions, the instructions being executable in one or more processor devices to cause the one or more processor to perform operations comprising identifying, at a computing device, a plurality of ozone gas generating devices that constitute the cohort, the plurality including at least a ceiling-mounted, an in-duct mounted and a portable ozone gas generating devices; continuously detecting, via at least one remote ozone gas sensor device located in a spatial area associated with the cohort in conjunction with one or more processors of the computing device, a concentration of ozone gas constituent within ozonated air of the spatial area; and instructing, by the one or more processors responsive to continuously detecting the concentration of the ozone gas constituent as being one of above and below a predetermined threshold concentration, at least one ozone gas generating device of the cohort to perform one of increasing and decreasing a rate of ozone gas generation associated therewith.

Embodiments described herein can be implemented using programmatic modules, through the use of instructions that are executable by one or more processors. A programmatic module can include a program, a sub-routine, a portion of a program, or a software component or a hardware component capable of performing one or more stated tasks or functions. As used herein, a programmatic module can exist on a hardware component independently of other modules or components, or can be a shared element of other modules, programs or machines.

One or more embodiments described herein provide that methods, techniques, and actions performed in an ozone generating device and system are performed programmatically, or as a computer-implemented method. Programmatically, as used herein, means through the use of code or computer-executable instructions. These instructions can be stored in one or more memory resources accessible to the ozone gas generating device.

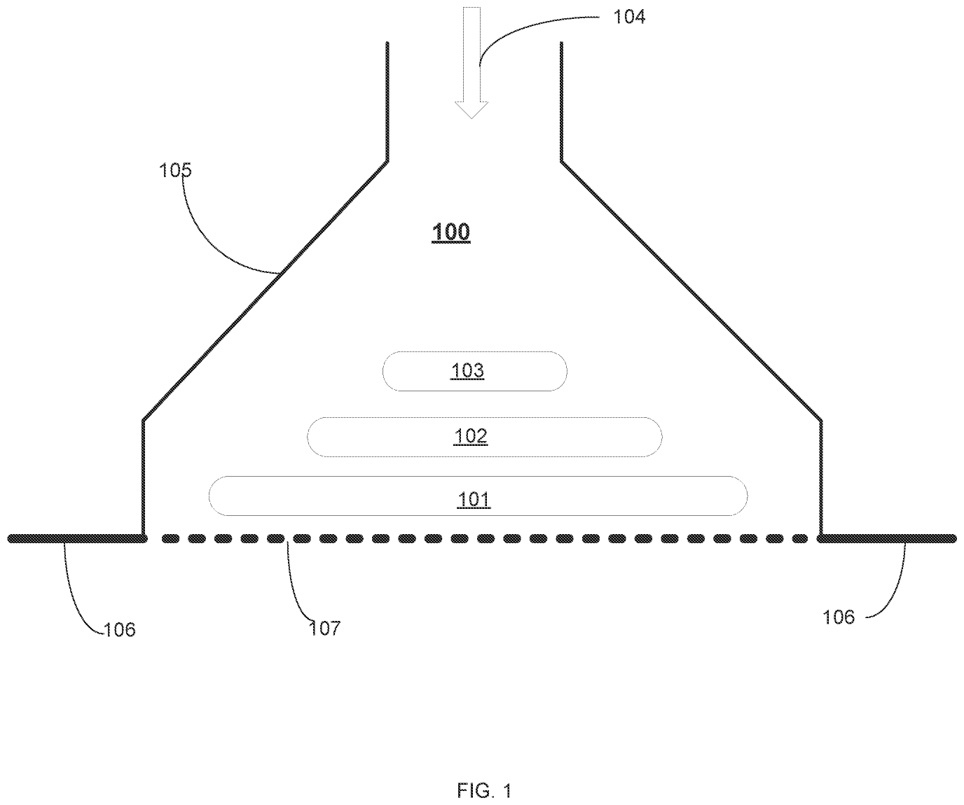

FIG. 1 illustrates, in an example embodiment, ceiling-mounted ozone gas generating device 100. In an embodiment, ozone generating device 100 includes housing 105, comprising a galvanized steel boot in some manifestations, having ingress port that receives inflow of ambient air stream 104. Ceiling-mounted ozone gas generating device 100 may include controller module 103 manifested in a printed circuit board electronic assembly and facilitating electronic interconnection with one or more optically irradiating lamps 102 providing ultraviolet irradiation in a wavelength of 185 nanometers (nm). Controller module 103 may be interconnected with external alternating current (AC) power supply mains providing an electrical power source. Power converters may be deployed in some cases to enable controller module 103 to also operate based on a direct current (DC) input. In DC operating embodiments, ozone generating device 100 operates at substantially constant-voltage as provided by the DC sources. Local ozone gas concentration sensors may be electrically interconnected to, or incorporated within, controller module 103.

One or more airflow pressure differential pressure-inducing fans or similar device(s) 101 may be deployed downstream of optically irradiating lamps 102 and be capable of forcibly directing and dispersing ozonated air through ceiling grill 107. In such downstream disposition, optically irradiating lamps 102 may be hidden, or at least partially hidden from observation via a vantage point below ceiling-mounted ozone gas generating device 100. The term “downstream” as used herein relates to passage of air and ozonated air from ingress 104 travelling through ceiling grill or vent 107 egress. It is contemplated that, in some embodiments, airflow pressure differential pressure-inducing fans 101 may be deployed upstream of optically irradiating lamps 102. In embodiments, ceiling grill 107 comprises a partially-airflow-obstructive grid configuration that functions to increase turbulence of the ozonated air, thereby providing more complete dispersion, and more thorough mixing, of egress ozonated air into ambient air of living space below ceiling-mounted ozone gas generating device 100. In some variations, ceiling-mounted ozone gas generating device 100 may be sized same as a standard ceiling tile, configured for visually seamless or geometrically compatible integration with suspended ceiling panel portions 106. In particular aspects, ceiling-mounted ozone gas generating device 100 may be within a 6 feet×6 feet suspended ceiling footprint generally considered a global standard for ceiling tiles.

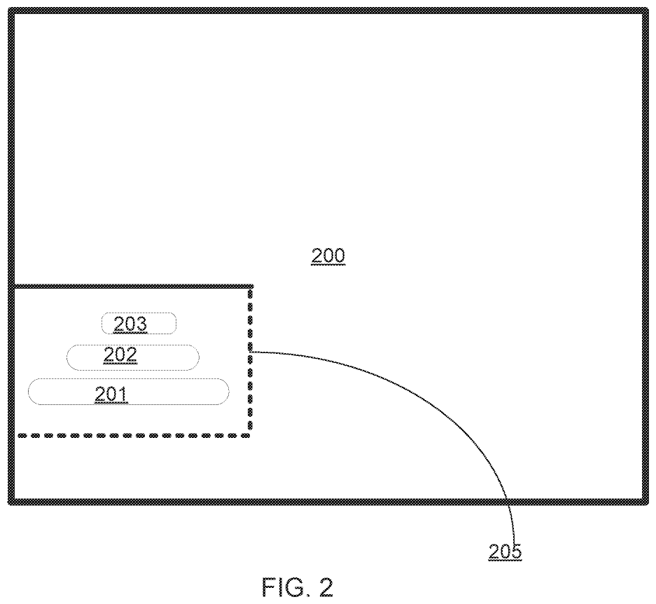

FIG. 2 illustrates, in an example embodiment, in-duct mounted ozone gas generating device 205 disposed within air supply duct 200. Air supply duct 200, in embodiments, may be a heating, ventilation and air conditioning (HVAC) supply infrastructure. In-duct mounted ozone gas generating device 205 may include controller module 203. In embodiments, electrically and functionally configured similar to controller module 103, and include one more local ozone gas sensors. In-duct mounted ozone gas generating device 205 may include optically irradiating lamps 202 and airflow pressure differential pressure-inducing fans 201 deployed to forcibly direct and disperse ozonated air via housing grill 205 into air supply duct 200 and then into living spaces.

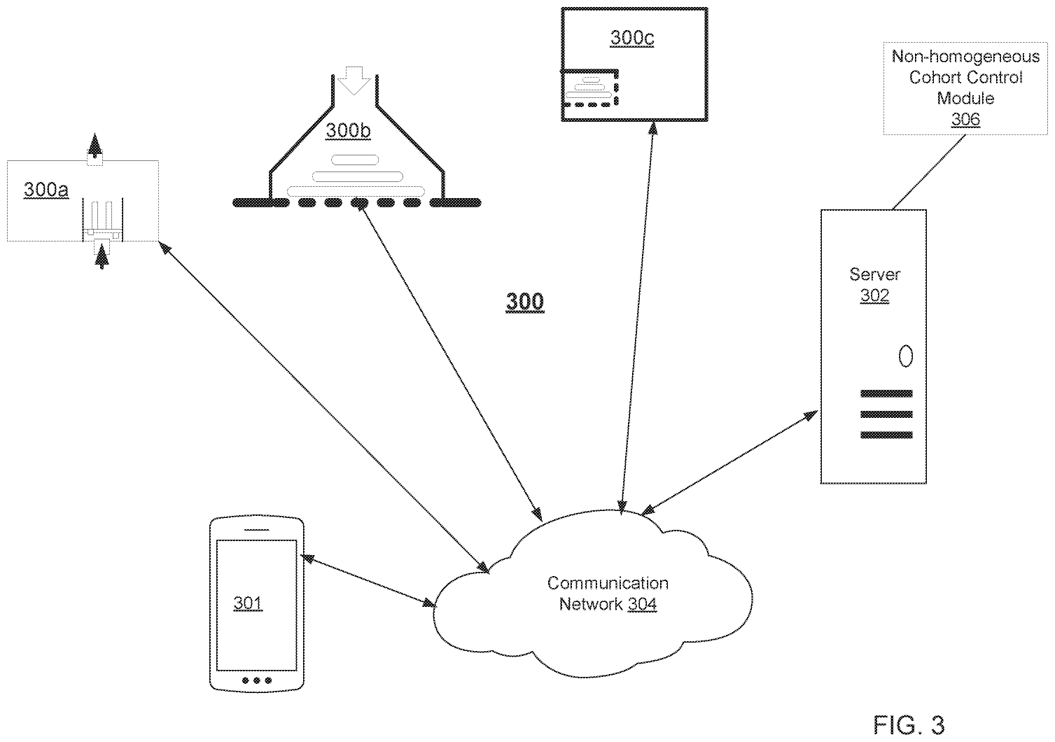

FIG. 3 illustrates an example embodiment system 300 of a non-homogeneous cohort of ozone gas generating devices 300a, 300b and 300c deployed in a communication network-based ozone generating system. In embodiments, ozone gas generating device 300a may be a portable ozone generating device, ozone gas generating device 300b a ceiling-mounted ozone gas generating device, and 300c an in-duct mounted ozone gas generating device, incorporating functionality and features described herein.

The non-homogeneous cohort of ozone gas generating devices 300a, 300b, 300c may be deployed within one or more given spatial areas, and further include any number of additional ozone gas generating devices. Non-homogeneous cohort of ozone gas generating devices 300a, 300b and 300c may be communicatively coupled with mobile device 301 which may be such as a mobile phone or tablet computing device. Ones of ozone generating devices 300a, 300b, 300c may also be communicatively coupled either directly with mobile device 301, and also in a cloud-based system as depicted, with server computing device 302 via communication network 304 which can, in some embodiments, be an internet or similar wide area or telecommunication-based protocols. In embodiments, mobile device 301 can be communicatively coupled with ozone gas generating devices 300a, 300b, 300c via wireless communication protocols including, but not limited to, Bluetooth, Wi-Fi, LoRa and/or RFID. In some embodiments, mobile device 301 may include a software application that provides a user interface for control of system 300 and also enable communication, either directly via wireless communication or within cloud-based system 300 via communication network 304, with ozone generating devices 300a, 300b, 300c in order to set or apply desired threshold values or acceptable ranges of ozone gas concentration, for instance as sensed by local ozone gas concentration sensor devices. In related embodiments, it is further contemplated that more than one mobile devices 301 may be deployed within system 300.

Server computing device 302 may be deployed in conjunction with mobile device 3011 within system 300. Server computing device 302 can include non-homogeneous cohort control module 306. In embodiments, it is contemplated that portions of the functionality as described herein for control of system 300 may be cooperatively enabled, and shared, between the software application executing on mobile device 301 and non-homogeneous cohort control module 306 of server computing device 302.

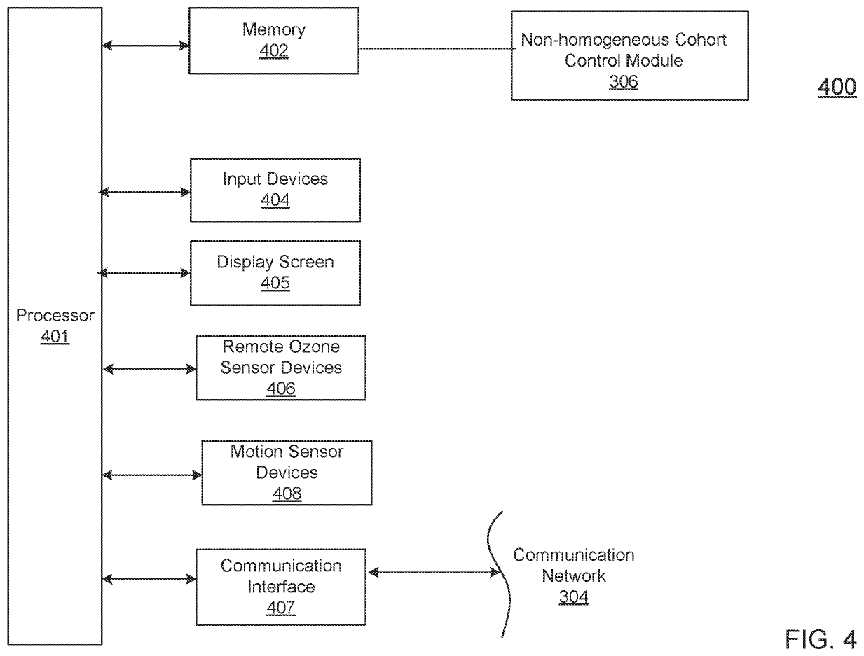

FIG. 4 illustrates, in an example embodiment, computing device architecture 400, in one embodiment as implemented at least partially in server computing device 302, for controlling non-homogeneous cohort of ozone gas generating devices 300a-c. Server computing device 302, in embodiments, may include processor 401, memory 402 and be communicatively interconnected with non-homogeneous cohort of ozone gas generating devices 300a-c via communication interface 407 that is communicatively coupled with communication network 304. Processor 401 can be implemented in an application specific integrated circuit (ASIC) device or field programmable gate array (FPGA) device, in some embodiments. Memory 402 may comprise any type of non-transitory system memory, storing instructions that are executable in processor 401, including such as a static random access memory (SRAM), dynamic random access memory (DRAM), synchronous DRAM (SDRAM), read-only memory (ROM), or any combination thereof.

Server computing device 302 can also be coupled with ozone gas concentration sensor devices, including ozone gas concentration sensor devices disposed within, in vicinity of, or remotely relative to the ones of non-homogeneous cohort of ozone gas generating devices 300a-c. In embodiments, server computing device 302 can include one or more user input devices 404, and also be communicatively coupled to remote motion sensor device (s) 406 to detect human presence, as may be inferred from motion, or lack thereof, with the areas surrounding the ones of non-homogeneous cohort of ozone gas generating devices 300a-c, for instance via wireless communication employing Wi-Fi or similar wireless communication protocols as described herein, configured in a cloud-connected network of sensors 406. In some embodiments, a cloud connected scheme of FIG. 4 can be implemented via cloud-based server computing device 302, with individual ones of non-homogeneous cohort of ozone gas generating devices 300a-c considered as respective Internet of Things (IoT) nodes within a given network arranged in a mesh or a star network configuration.

Server computing device 302 may also include capability for communicatively receiving and transmitting wireless communication signals, including but not limited to any of Bluetooth, Wi-Fi, LoRa, RFID, and global positioning system (GPS) signals, and incorporate communication interface 407 for communicatively coupling to communication network 304, such as by sending and receiving data transmissions.

Non-homogeneous cohort control module 306, in embodiments, can be constituted of computer processor-executable code accessibly stored in memory 402 that are executable in processor 401, to accomplish ozone gas generation functionality as described herein, associated with usage or deployment of non-homogeneous cohort of ozone gas generating devices 300a-c. In one embodiment, the software instructions or programs, including any updates thereof, constituting ozone generator logic module 310 can be downloaded to memory 202 by accessing and downloading, via communication network 304, from a remote server computing device, including from server 302, or from mobile computing device 301 via wireless communication protocols as described herein.

Non-homogeneous cohort control module 306, in embodiments, enables deployment of ozone gas generating devices 3-00a-c within ozone gas generating system 300 and includes, in non-transitory memory 402, logic instructions that are executable in processor 401. The instructions when executed by processor 401 cause the processor to perform operations comprising receiving a stream of ambient air that includes gaseous oxygen, generating ozone gas in accordance with applying ultraviolet (UV) irradiation provided in a wavelength of 185 nanometer (nm) to at least a portion of the gaseous oxygen constituted in the stream of ambient air, the UV irradiation provided via an optical lamp module powered by a direct current (DC) battery source, or via AC source from a power mains source producing a modified air stream of ozonated air having a higher concentration of ozone gas as compared with a trace concentration of ozone gas that is constituted in the incoming stream of ambient air via ingress ports of respective one of ozone gas generating devices 3-00a-c.

Ozone generator logic module 310 of controller module 103, in some embodiments, also includes, as configured within non-transitory memory 402, logic instructions that are executable in processor 401 to adjust the rate of generating ozone gas based on local and remote ozone sensor devices 406. In embodiments, a plurality of motion sensors 408, or occupancy sensors, can be deployed within the spatial area, and be communicatively coupled to server computing device 203 and to ozone gas generating devices 300a-c. In one embodiment in accordance with a heightened safety protocol, remote motion sensor devices can be used to detect that no human persons or living creatures are active and within the surroundings, such as an enclosed room in which the ozone gas generating device is located, before switching to a second mode of operation having increased rate of generation or production of ozone gas. In embodiments, remote motion sensor 408 may be a proximity-based sensor or may comprise similar sensors deployed to detect or infer presence or absence of living occupants within the spatial area or surroundings. For example, besides a motion sensor, ultrasonic sensors that detect shifts or changes in sound waves that might be associated with presence of living occupants may be deployed to infer presence, or absence, of living occupants within a given space. Infrared radiation sensors that detect heat generated from the living occupants can also be deployed to infer presence, or absence, of living occupants within a given habitable space. Camera imaging could be deployed, in some embodiments, to detect or infer presence or absence of living occupants. A second, or alternate, modified airstream generated in this higher order, or “turbocharged”, mode of operation can comprise a higher concentration of ozone gas than the first modified airstream, and optionally be generated with a higher flowrate of exhausting as compared with the first modified airstream. In this manner, under conditions where the ozone gas concentration level within a given living space is lower than a desired threshold level and no living being is active or occupying the space, a higher rate of production of ozone gas can be deployed within a given time period for safe dissemination into the surroundings while avoiding potentially adverse effects on living occupants in the space. In embodiments, a safe and desired threshold level of ozone gas concentration that provides effective anti-viral and anti-bacterial functions, as sensed by either local or remote ozone gas sensor devices 406, may be in the range between 50 parts per billion (ppb) and 100 ppb, though it is contemplated that other ranges or values can be implemented.

In some embodiments, computing device architecture 400 incorporates an artificial intelligence machine learning-based system as described herein for controlling a non-homogeneous cohort of ozone gas generating devices 300a-c. The term cohort, as referred to herein, refers to a group of ozone gas generating devices deployed within one or more given spatial areas for common, or individual, purposes of generating ozone gas within ambient air of the spatial areas. The spatial area may be a room, a hall, an enclosed or partially enclosed area, and can be defined or designated in terms of location coordinates, either in local or global (x,y) coordinates that define a boundary or perimeter enclosing the spatial area. The term non-homogeneous as applied herein to a non-homogeneous cohort of devices refers to ozone gas generating devices which inherently have different operating characteristics. The different operating characteristics may be such as, but not limited to, capability for rate of generation of ozone gas, physical form factors of the ozone gas generating devices, their modes of attachment, or non-attachment in the case of the portable devices, to physical infrastructure in which they operate, their expected or typical operating locations within the physical infrastructure, and their input electrical power characteristics, among others. In the embodiment depicted in FIG. 1, ozone gas generating devices 300a, 300b, 300c collectively may be referred to as such a non-homogeneous cohort of may be deployed within one or more given spatial areas.

Processor 201 uses executable instructions stored in AI, or machine learning, neural network module herein to detect, via at least one of ozone gas sensor devices 406a located in spatial areas associated with the non-homogeneous cohort, a concentration of ozone gas constituent of ambient air within the spatial area. In embodiments, the concentration of ozone gas constituent of ambient air may be detected based on at least partly upon using a trained machine learning model in conjunction with the plurality of ozone gas sensor devices 406.

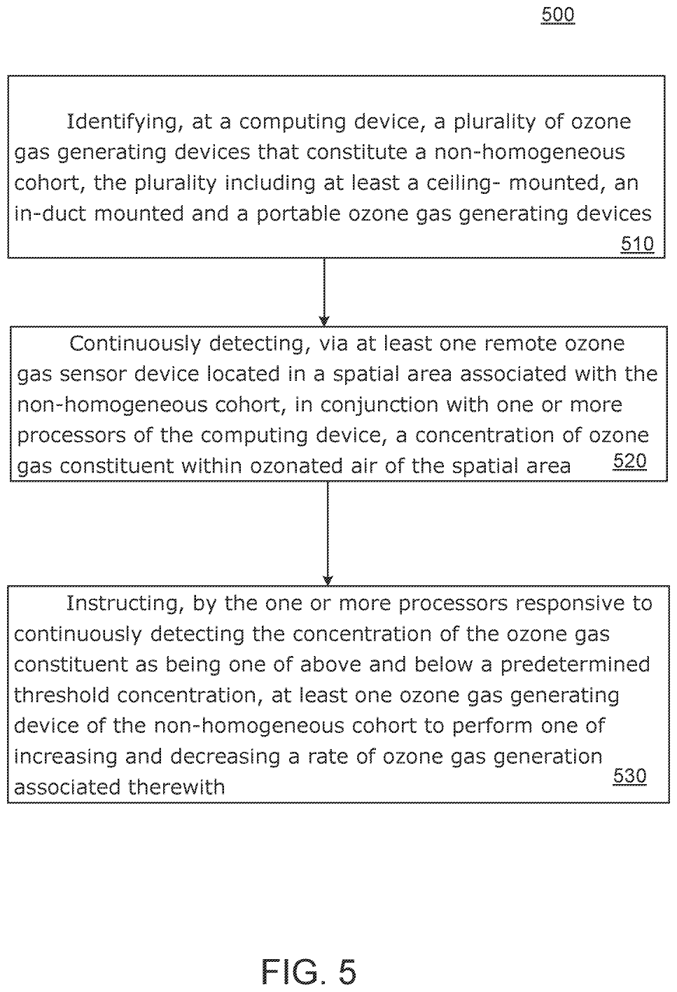

FIG. 5 illustrates, in an example embodiment, method 500 of operation in cloud-based control of a non-homogeneous cohort of ozone gas generating devices 300a-c. Examples of method steps described herein are related to deployment and use of ozone generating device 101 as described herein. According to one embodiment, the techniques are performed in processor 401 executing one or more sequences of software logic instructions that constitute non-homogeneous cohort control module 306. In embodiments, instructions constituting non-homogeneous cohort control module 306 may be read into memory 402 from machine-readable medium, such as memory storage devices. Executing the instructions of non-homogeneous cohort control module 306 stored in memory 402 causes processor 401 to perform the process steps described herein. In alternative implementations, at least some hard-wired circuitry may be used in place of, or in combination with, the software logic instructions to implement examples described herein. Thus, the examples described herein are not limited to any particular combination of hardware circuitry and software instructions.

In embodiments, a safe and desired threshold level of ozone gas concentration that provides effective anti-viral and anti-bacterial functions, as sensed by either local or remote ozone gas sensor devices 406, may be in the range between 5 parts per billion (ppb) and 1,000 ppb. However, it is contemplated that other ranges or values can be deployed; for instance, in a range from 50 ppb to 500 ppb of ozone gas concentration.

At step 510, identifying, at a computing device, a plurality of ozone gas generating devices that constitute the non-homogeneous cohort, the plurality including at least a ceiling-mounted, an in-duct mounted and a portable ozone gas generating devices.

In a further variation, using one or more remote ozone gas concentration sensor device(s) 406, the condition external to the housing can be determined as a concentration of ozone gas being below a predetermined threshold concentration, for instance in a range of 50 to 500 ppb, within a predetermined area around the housing of ozone gas generating device 101.

At step 520, continuously detecting, via at least one remote ozone gas sensor device located in a spatial area associated with the cohort in conjunction with one or more processors of the computing device, a concentration of ozone gas constituent within ozonated air of the spatial area. In one embodiment, continuously detecting the concentration of ozone gas constituent within ozonated air of the spatial area is performed in accordance with continuously sampling, at either a predetermined time interval or a variable interval, said concentration. In some aspects, the variable interval can vary within a predetermined range of time intervals.

At step 530, instructing, by the one or more processors responsive to continuously detecting the concentration of the ozone gas constituent as being one of above and below a predetermined threshold concentration, at least one ozone gas generating device of the cohort to perform one of increasing and decreasing a rate of ozone gas generation associated therewith

In some embodiments, the cohort of ozone gas generating devices are communicatively coupled to the computing device within a cloud communication network. Ceiling mounted ozone gas generating device 300b, in some aspects, comprises an optical lamp module including a plurality of optical lamps and a fan apparatus arranged for forcibly dispersing ozonated air in a downward direction relative to a ceiling portion of an at least partially enclosed building wherein the ceiling mounted ozone gas generating device is housed. In some variations, the ceiling portion of the at least partially enclosed building comprises a ceiling tile of a predetermined size within a suspended ceiling configuration, and the ozone gas generating device is mounted generally within a footprint of the ceiling tile.

In some embodiments, the optical lamp module is disposed either upstream and downstream, relative to the incoming stream of air, of the fan apparatus. In some aspects, when disposed in the upstream position, the optical lamp module is at least partially obscured by the fan apparatus when viewed by an observer on the floor below the ceiling portion. In some embodiments, the in-duct mounted ozone generating device comprises an optical lamp module that includes a plurality of optical lamps and a fan apparatus arranged for forcibly dispersing ozonated air along at least a portion of an air supply duct that houses the in-duct mounted ozone generating device.

In some variations, in-duct mounted ozone generating device 300c comprises an optical lamp module including a plurality of optical lamps and a fluid flow sensor device, such as but not limited to, one or more fluid flow pressure-actuated sensor devices that detect passage of air along the duct. In some aspects, the optical lamp module including the plurality of optical lamps is operationally switched on for generation of ozonated air upon detecting passage of air along the duct that exceeds a predetermined, or a threshold, flow rate. The remote ozone gas sensor devices may be located, in some embodiments, within the spatial area associated with a given cohort of ozone generating devices, the spatial area including one or more at least partially enclosed buildings which may be either adjoining or disparately situated relative to one another. For instance, in some aspects the spatial area associated with the cohort can multiple buildings, or parts of buildings, within a campus infrastructure.

In yet another embodiment, the method includes continuously detecting the concentration of ozone gas constituent of ozonated air at least partly using a trained machine learning model in conjunction with the plurality of remote ozone gas sensor devices. The trained machine learning model, in some embodiments, may be trained via a training process that includes receiving a plurality of input datasets at respective ones of a plurality of input layers of a neural network, the neural network being instantiated in the one or more processors and having an output layer interconnected to the plurality of input layers via a set of intermediate layers, each of the plurality of input datasets comprising an input attribute associated with ones of the plurality of ozone gas generating devices, ones of the set of intermediate layers being configured in accordance with an initial matrix of weights; and training the neural network in accordance with the respective ones of the plurality of input layers based at least in part upon recursively adjusting the initial matrix of weights by back propagation in generating, at the output layer, an output attribute in accordance with diminishment of an error matrix computed at the output layer of the neural network.

In some aspects, the plurality of input datasets can comprise one or more of ozone gas generation capacity in regular mode of operation, location coordinates defining external boundaries or perimeter of a given spatial area, coordinate location of ozone gas generating device within the spatial area, model identification of ozone gas generating device, device operational reliability metrics, device wireless communication reliability metrics and device historical, cumulative ozone gas generating metrics. The output attribute, in some embodiments, comprises a desired concentration of ozone gas as constituted in ozonated air of the spatial area.

Although embodiments are described in detail herein with reference to the accompanying drawings, it is contemplated that the disclosure herein is not limited to only such literal embodiments. As such, many modifications and equivalents of the ozone gas generating devices and variations in sequence of the method steps in conjunction with varying combinations of user interface features disclosed herein will be apparent to practitioners skilled in this art. Accordingly, it is intended that the scope of the invention be defined by the following claims and their equivalents. Furthermore, it is contemplated that a particular feature described either individually or as part of an embodiment can be combined with other individually described features, or parts of other embodiments described herein. Thus, absence of any described particular combinations of such does not preclude the inventor from claiming rights to such combinations.

What is claimed is:

1. A method of controlling a non-homogeneous cohort of ozone gas generating devices, the method comprising:
identifying, at a computing device, a plurality of ozone gas generating devices that constitute the non-homogeneous cohort, the plurality including at least a ceiling-mounted, an in-duct mounted and a portable ozone gas generating devices;
continuously detecting, via at least one remote ozone gas sensor device located in a spatial area associated with the non-homogeneous cohort, in conjunction with one or more processors of the computing device, a concentration of ozone gas constituent within ozonated air of the spatial area; and
instructing, by the one or more processors responsive to continuously detecting the concentration of the ozone gas constituent as being one of above and below a predetermined threshold concentration, at least one ozone gas generating device of the non-homogeneous cohort to perform one of increasing and decreasing a rate of ozone gas generation associated therewith.

2. The method of claim 1 wherein the non-homogeneous cohort of ozone gas generating devices are communicatively coupled to the computing device within a cloud communication network.

3. The method of claim 1 wherein the at least one ceiling mounted ozone gas generating device comprises an optical lamp module including a plurality of optical lamps and a fan apparatus arranged for forcibly dispersing ozonated air in a downward direction relative to a ceiling portion of an at least partially enclosed building wherein the ceiling mounted ozone gas generating device is housed.

4. The method of claim 3 wherein the ceiling portion of the at least partially enclosed building comprises a ceiling tile of a predetermined size, and the at least one ceiling mounted ozone gas generating device further comprises a mounted configuration that is generally coincident with a footprint of the ceiling tile.

5. The method of claim 3 wherein the optical lamp module is disposed at one of upstream and downstream of the fan apparatus.

6. The method of claim 5 wherein, in the upstream disposition, the optical lamp module is at least partially obscured by the fan apparatus from a view relative to an observer distally situated below the ceiling portion.

7. The method of claim 1 wherein the in-duct mounted ozone generating device comprises an optical lamp module including a plurality of optical lamps and a fan apparatus arranged for forcibly dispersing ozonated air along at least a portion of an air supply duct that houses the in-duct mounted ozone generating device.

8. The method of claim 1 wherein the in-duct mounted ozone generating device comprises an optical lamp module including a plurality of optical lamps and a fluid flow sensor device that detects passage of air along the duct.

9. The method of claim 8 wherein the optical lamp module including the plurality of optical lamps is operationally switched on for generation of ozonated air responsive to detecting passage of air along the duct that exceeds a predetermined flow rate.

10. The method of claim 1 wherein the at least one remote ozone gas sensor device comprises a plurality of remote ozone gas sensor devices located within the spatial area associated with the non-homogeneous cohort, the spatial area including an at least partially enclosed building.

11. The method of claim 10 wherein the spatial area associated with the non-homogeneous cohort includes multiple buildings within a campus infrastructure that includes the at least partially enclosed building.

12. The method of claim 1 further comprising continuously detecting the concentration of ozone gas constituent of ozonated air at least partly using a trained machine learning model in conjunction with the plurality of remote ozone gas sensor devices.

13. The method of claim 12 further comprising producing the trained machine learning model via a training process comprising:
receiving a plurality of input datasets at respective ones of a plurality of input layers of a neural network, the neural network being instantiated in the one or more processors and having an output layer interconnected to the plurality of input layers via a set of intermediate layers, each of the plurality of input datasets comprising an input attribute associated with ones of the plurality of ozone gas generating devices, ones of the set of intermediate layers being configured in accordance with an initial matrix of weights; and
training the neural network in accordance with the respective ones of the plurality of input layers based at least in part upon recursively adjusting the initial matrix of weights by back propagation in generating, at the output layer, an output attribute in accordance with diminishment of an error matrix computed at the output layer of the neural network.

14. The method of claim 13 wherein the plurality of input datasets comprise one or more of: ozone gas generation capacity in regular mode of operation, location coordinates defining external boundaries or perimeter of a given spatial area, coordinate location of ozone gas generating device within the spatial area, model identification of ozone gas generating device, device operational reliability metrics, device wireless communication reliability metrics and device historical, cumulative ozone gas generating metrics.

15. The method of claim 13 wherein the output attribute comprises a desired concentration of ozone gas as constituted in ozonated air of the spatial area.

16. The method of claim 1 wherein the at least one ozone gas generating device of the non-homogeneous cohort performs ozone gas generation in accordance with applying ultraviolet (UV) irradiation provided in a wavelength of 185 nanometer (nm) to at least a portion of the gaseous oxygen constituted in an incoming stream of air to produce ozonated air, the ozonated air having a higher concentration of ozone gas than the incoming stream of air.

17. A computing device comprising:
a processor; and
a non-transitory memory including instructions, the instructions when executed by the processor causing the processor to perform operations comprising:
identifying a plurality of ozone gas generating devices that constitute a non-homogenous cohort, the plurality including at least a ceiling-mounted, an in-duct mounted and a portable ozone gas generating devices;
continuously detecting, via at least one remote ozone gas sensor device located in a spatial area associated with the non-homogeneous cohort, in conjunction with one or more processors of the computing device, a concentration of ozone gas constituent within ozonated air of the spatial area; and
instructing, by the one or more processors responsive to continuously detecting the concentration of the ozone gas constituent as being one of above and below a predetermined threshold concentration, at least one ozone gas generating device of the non-homogeneous cohort to perform one of increasing and decreasing a rate of ozone gas generation associated therewith.

18. A non-transitory computer-readable memory storing instructions, the instructions being executable in one or more processor devices to cause the one or more processor to perform operations comprising:
identifying, at a computing device, a plurality of ozone gas generating devices that constitute a non-homogeneous cohort, the plurality including at least a ceiling-mounted, an in-duct mounted and a portable ozone gas generating devices;
continuously detecting, via at least one remote ozone gas sensor device located in a spatial area associated with the non-homogeneous cohort, in conjunction with one or more processors of the computing device, a concentration of ozone gas constituent within ozonated air of the spatial area; and
instructing, by the one or more processors responsive to continuously detecting the concentration of the ozone gas constituent as being one of above and below a predetermined threshold concentration, at least one ozone gas generating device of the non-homogeneous cohort to perform one of increasing and decreasing a rate of ozone gas generation associated therewith.
